(19) United States
(12) Patent Application Publication
(10) Pub. No.: US 2021/0058695 A1
(43) Pub. Date: Feb. 25, 2021

(54) BONE CONDUCTION AUDIO DEVICE

(71) Applicant: Good Sportsman Marketing, LLC, Irving, TX (US)

(72) Inventors: Howard Unger, Henderson, NV (US); Thomas Rucci, Henderson, NV (US)

(73) Assignee: Good Sportsman Marketing, LLC, Irving, TX (US)

(21) Appl. No.: 16/867,530

(22) Filed: May 5, 2020

Related U.S. Application Data
(60) Provisional application No. 62/822,753, filed on Mar. 22, 2019.

(51) Int. Cl. H04R 1/10; H04R 1/26

(52) U.S. Cl. CPC H04R 1/1075; H04R 2460/13; H04R 1/265

(57) ABSTRACT

A bone conduction audio device transmits sound to a user via a skeletal pathway to prevent isolation of the user in environments where hearing protection is desired or required. A variety of hearing protection devices are compatible with the bone conduction audio device. A first and second microphone may respectively record different sources of sound, which are transmitted to a user's ears, an auxiliary device, or both.

### CROSS-REFERENCE TO RELATED APPLICATION

This application claims priority to U.S. Provisional Patent Application No. 62/822,753, filed Mar. 22, 2019.

### BACKGROUND OF THE INVENTION

### 1. Field of the Invention

The invention relates to hearing protection headsets and in particular to a bone conduction audio device.

### 2. Related Art

Hearing protection headsets are often worn to attenuate sounds that may be harmful to a user's hearing or that a user does not wish to hear. Typically, such sound attenuation is achieved by impeding the transmission of sound, masking or cancelling sounds with other sounds, or both.

From the discussion that follows, it will become apparent that the present invention addresses the deficiencies associated with the prior art while providing numerous additional advantages and benefits not contemplated or possible with prior art constructions.

### SUMMARY OF THE INVENTION

A bone conduction audio device is disclosed herein. As will be described below, a bone conduction audio device will typically transmit ambient sound to a user via a bone or skeletal conduction pathway. A bone conduction audio device may be used by users desiring or requiring hearing protection devices as well as by hearing impaired users to improve the reception or hearing of ambient sound.

Various embodiments of the bone conduction audio device and methods therefor are disclosed herein. For instance, in one exemplary embodiment a bone conduction audio device comprises one or more microphones that record sound, one or more conduction transducers that transmit the sound to a user via a skeletal structure of the user, and a support that secures the conduction transducers in engagement with the skeletal structure of the user when the bone conduction audio device is worn by the user. The user's ears are not obstructed by the conduction transducers.

The bone conduction audio device may include an auxiliary microphone that records a user's voice. An I/O device may transmit the user's voice to an auxiliary device. A filter may attenuates one or more frequency ranges in the sound. A filter may also or alternatively attenuate the sound when the sound is above a particular decibel level. A filter may also amplify one or more frequency ranges in the sound. One or more hearing protection devices may be provided as well.

In another exemplary embodiment, a bone conduction audio device comprises one or more microphones that record sound, one or more conduction transducers that transmit the sound to a user via a skeletal structure of the user, and a support that secures the conduction transducers in engagement with the skeletal structure of the user when the bone conduction audio device is worn by the user. One or more mounts are also provided for holding one or more hearing protection devices. Similar to above, the user's ears are not obstructed by the conduction transducers.

The bone conduction audio device may include an auxiliary microphone that records a user's voice. An I/O device may transmit the user's voice to an auxiliary device. A filter may attenuates one or more frequency ranges in the sound. A filter may also or alternatively attenuate the sound when the sound is above a particular decibel level. A filter may also amplify one or more frequency ranges in the sound. It is noted that the support may comprise a resilient structure.

Various methods with respect to a bone conduction audio device are disclosed herein as well. For example, a method for providing auditory perception with a bone conduction audio device is disclosed, with such method comprising providing one or more conduction transducers that transmit sound to a user via a skeletal structure of the user. A support secures the conduction transducers in engagement with the skeletal structure of the user when the bone conduction audio device is worn by the user.

The method includes capturing sound via one or more microphones and transmitting the sound to the user with the conduction transducers via the skeletal structure of the user. The user's ears are not obstructed by the conduction transducers. The sound may be obstructed at the user's ears with one or more hearing protection devices.

An auxiliary microphone that records a user's voice may be provided as well. The user's voice may be transmitted to an auxiliary device with an I/O device. The sound may be attenuated with a filter when the sound is above a particular decibel level. Alternatively or in addition, one or more frequency ranges in the sound may be amplified with a filter.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1A is a perspective view of an exemplary bone conduction audio device;

FIG. 1B is a side view of an exemplary bone conduction audio device in an environment of use, the opposite side view being a mirror image thereof; and

FIG. 2 is a block diagram of an exemplary bone conduction audio device.

### DETAILED DESCRIPTION OF THE INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

The bone conduction audio device herein allows a user to hear ambient sound, including the sound of activity, alarms or notifications, communications, or other occurrences within their environment via bone conduction. The ability to hear ambient sound in this manner is advantageous at least for the reason that it prevents a user from being isolated from their environment. Also, because ambient sound is available via bone conduction, a user's ears are unencumbered and can be freely adorned with one or more hearing protection devices to protect their hearing. In addition, the decibel level of ambient sound can be amplified such as to aid hearing impaired users.

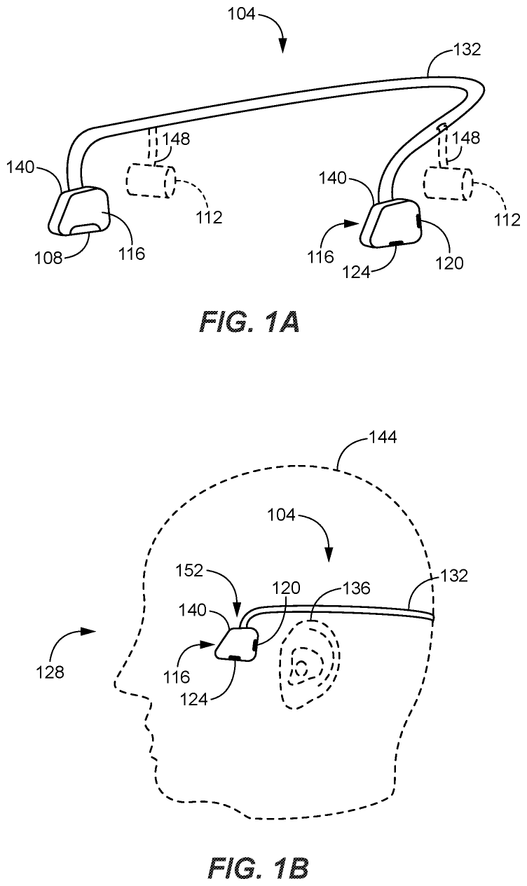

FIGS. 1A and 1B illustrate an exemplary bone conduction audio device 104 as may be worn by a user 128. As can be seen, a bone conduction audio device 104 may comprise one or more conduction transducers 116 and one or more ambient microphones 120. One or more power sources 108, such as batteries will typically be included as well.

In operation, ambient sounds may be recorded at one or more ambient microphones 120 and transmitted to a user's ears 136, such as to the user's inner ear, by one or more conduction transducers 116.

A conduction transducer 116 transmits ambient sound through the user's skeletal structure. Because a bone or skeletal conduction pathway is relied upon for ambient sound transmission, the user's ears 136 are unencumbered and not covered, blocked, or otherwise obstructed by the bone conduction audio device 104. This allows various hearing protection devices 112 to be readily adorned at a user's ears 136 without impairment of a user's ability to hear.

To illustrate, a user 128 may wear one or more earplugs, earmuffs, other hearing protection devices 112, or various combinations thereof while retaining the ability to hear ambient sound, such as the sound of activity, alarms or notifications, communications, or other occurrences within their environment, which would otherwise be attenuated by the hearing protection devices.

In this manner, the user 128 is not isolated from their environment and can remain audibly aware of and respond to sounds in their environment, including voices or alarms, while remaining protected. Hearing protection devices 112 or combinations of hearing protection devices of even the highest noise reduction rating can entirely block or isolate the ear canal, outer ear, or both, providing maximized hearing protection while the user retains the ability to hear ambient sound via the bone conduction audio device 104.

It is contemplated that, in some embodiments, one or more hearing protection devices 112 may be provided with or be part of a bone conduction audio device 104. For example, one or more earplugs, one or more earmuffs, or both may be mounted to a support 132 or other portion of a bone conduction audio device 104 via one or more mounts 148. It is contemplated that these hearing protection devices 112 may be removable from a bone conduction audio device 104, so as to permit them to be maintained or replaced as desired.

As indicated by the broken line depiction thereof, mounts 148 and mounting of hearing protection devices 112 to a bone conduction audio device 104 is optional and may not be provided in all embodiments. In addition, hearing protection devices 112 are optional and need not always be used or provided with a bone conduction audio device 104. As described above for example, a bone conduction audio device 104 may be used to amplify ambient sound for hearing impaired users or other user not wearing a hearing protection device 112.

In some embodiments, a bone conduction audio device 104 may optionally include one or more auxiliary microphones 124. As will be described further below, an auxiliary microphone 124 will typically be used to record a user's voice for an auxiliary device 224, such as a smartphone, two-way radio, or other communication device.

A bone conduction audio device 104 may comprise one or more enclosures or housings 140. As shown in FIGS. 1A and 1B, two housings 140 are provided with one housing at each side of the user's head 144. This allows recording and transmission of ambient sound in stereo via one or more ambient microphones 120 and one or more conduction transducers 116 at each side of the user's head 144. It will be understood that ambient sound may be recorded and transmitted in mono or stereo or other spatial sound.

Various components of a bone conduction audio device 104 may be installed or housed in an enclosure or housing 140. As shown in the exemplary embodiment of FIGS. 1A and 1B for instance, a power source 108 and conduction transducer 116 are secured to a housing 140. Similarly, one or more microphones 120, 124 may be secured to a housing 140. A housing 140 may enclose and protect such components as well, while providing avenues for one or more microphones 120, 124 to receive sound if necessary. For example, a housing 140 may be an enclosure having one or more apertures to allow transmission of sound to a microphone 120, 124.

It is contemplated that, in embodiments with multiple housings 140, each housing may support the same or various sets of components. For example, a first housing 140 may house a conduction transducer 116 and one or more microphones 120, 124, while a second housing houses a power source 108 in addition to a conduction transducer and one or more microphones. In another exemplary embodiment, only one housing 140 may have one or more microphones 120, 124. In yet another exemplary embodiment, only one housing 140 may have one or more conduction transducers 116. A wired or wireless connection may transmit audio signals, power, or both between housings 140.

One or more supports 132 may be provided to secure a bone conduction audio device 104 to a user 128. As shown in FIGS. 1A and 1B for example, a support 132 secures the bone conduction audio device 104 to a user's head 144. Typically, a support 132 will secure a conduction transducer 116 such that the conduction transducer engages the skeletal or other rigid structure of a user 128. As shown for example, one or more conduction transducers 116 may be held such that they engage the user's cheekbones 152. This allows sound to be transmitted to and through the user's skin and skeletal structure to the user's ears 136 when a conduction transducer 116 vibrates to transmit such sound.

Though shown as a band extending horizontally around a back end of a user's head 144, it is contemplated that a support 132 may extend over or under the user's head, in some embodiments. A support 132 may comprise one or more biasing devices or resilient structures which press one or more conduction transducers 116 against a user's skeletal structure. In addition to a band, a support 132 may be an ear hook, hat, helmet, headband, or other headwear.

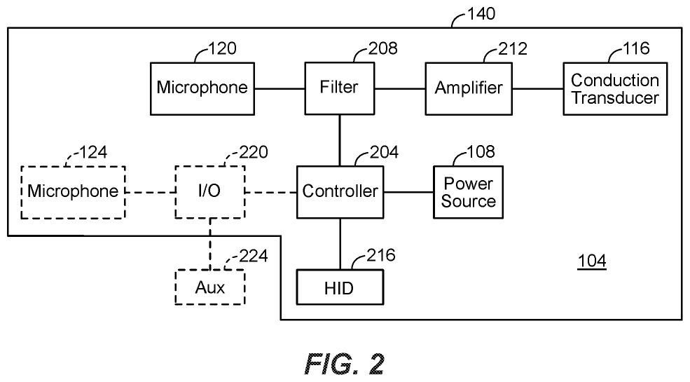

FIG. 2 illustrates components of an exemplary bone conduction audio device 104. As described above, a bone conduction audio device 104 may comprise one or more power sources 108, one or more conduction transducers 116, and one or more ambient microphones 120 for recording ambient sound. As shown in FIG. 2, a bone conduction audio device 104 may also include one or more amplifiers 212, which provide amplification of ambient sounds recorded by an ambient microphone 120 sufficient to vibrate or otherwise excite a conduction transducer 116 to transmit audible sound to a user via the skeletal pathway.

One or more CODECs or filters 208 may be provided to suppress or otherwise attenuate ambient sound recorded at one or more ambient microphones 120. Typically, a filter will suppress ambient sound having one or more particular characteristics. For example, a filter 208 may reduce the decibel level of ambient sound that is above a predefined decibel threshold, within a particular frequency range, or both. In some embodiments, such suppression may occur for a predetermined period of time. This is advantageous for suppressing momentary sounds such as gun shots.

In addition or alternatively, suppression may occur by lowering the decibel level of ambient sound to maintain a particular decibel level. For example, a filter 208 may constantly reduce the decibel level of ambient sound above a particular decibel level to maintain ambient sound at a constant decibel level. It is noted that a constant decibel level may be a range of decibel levels in some embodiments.

To illustrate, a filter 208 may allow ambient sound at 85 decibels to be transmitted to a user without suppression. If the ambient sound should reach 95 decibels or higher, the filter 208 may suppress the decibel level such that the ambient sound is between 85 and 90 decibels when transmitted to the user.

It is noted that a filter 208 may also attenuate or amplify frequency ranges of sound. For example, a filter 208 may include one or more high pass, low pass, or bandpass filters. A filter's suppression of decibel levels and attenuation or amplification of various frequency ranges may be user-adjustable in one or more embodiments, as described below.

Though shown as a separate component, it is noted that a filter 208 may be integral with an amplifier 212 or other component in one or more embodiments. In addition, it is noted that in embodiments without a filter 208, an ambient microphone 120 may be directly connected to an amplifier 212.

In some embodiments, one or more I/O devices 220 may be included to provide interoperability with one or more auxiliary devices 224, such as two-way radios, radio receivers, smartphones, telephones, media players, or other audio devices. An I/O device 220 may transmit audio signals, receive audio signals, or both via a wired or wireless connection. Some exemplary I/O devices 220 include BLUETOOTH or other wireless transceivers and audio ports.

In operation, a user's voice or other auxiliary sound recorded by an auxiliary microphone 124 may be transmitted to one or more auxiliary devices 224 via an I/O device 220. In addition, ambient sound recorded by an ambient microphone 120 may be transmitted to one or more auxiliary devices 224 via an I/O device 220. It is contemplated that auxiliary sound may also or alternatively be audio output from an auxiliary device 224 that is received by a bone conduction audio device 104 via an I/O device 220. Auxiliary sound from one or more sources may be transmitted to a user along with ambient sound from one or more ambient microphones 120 via one or more conduction transducers 116 in some embodiments.

As indicated by the broken line depiction thereof, auxiliary microphones 124, I/O devices 220, and interoperability with auxiliary devices 224 is optional and may not be provided in all embodiments of the bone conduction audio device 104.

One or more controllers 204 may be provided to control operation of various aspects of a bone conduction audio device 104. For example, a controller 204 may provide power management functionality, control decibel levels, control ambient sound suppression or filtering, or various subsets thereof. In one or more embodiments, a controller 204 may comprise a microcontroller, processor, integrated circuit, or the like. Control of decibel levels, ambient sound suppression, or ambient sound filtering may be based on user input.

One or more human interface devices 216 may be included to receive such user input. Some exemplary human interface devices 216 include buttons, switches, knobs, touch pads/screens, and other controls. A human interface device 216 may be manipulated or otherwise engaged to activate or deactivate or adjust various functions of a bone conduction audio device 104. For example, a human interface device 216 may be engaged to adjust the decibel level of ambient sound or auxiliary sound at one or more amplifiers 208, conduction transducers 116, or both. A human interface device 216 may also be engaged to adjust one or more decibel level thresholds or ranges associated with ambient sound suppression. In addition, a human interface device 216 may be engaged to adjust high, low, or bandpass filtering thresholds or ranges.

Though shown generally in a star topology, it is contemplated that the components of a bone conduction audio device 104 may be interconnected in various ways, including via a start, hub, or other topology, serial or parallel connections, or various subsets thereof. Though shown as connected via a single interconnects, it will be understood that various components of a bone conduction audio device 104 may be connected to one or more other components via one or more interconnects.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A bone conduction audio device comprising:
one or more microphones that record sound;
one or more conduction transducers that transmit the sound to a user via a skeletal structure of the user; and
a support that secures the one or more conduction transducers in engagement with the skeletal structure of the user when the bone conduction audio device is worn by the user;
wherein the user's ears are not obstructed by the one or more conduction transducers.

2. The bone conduction audio device of claim 1, further comprising an auxiliary microphone that records a user's voice.

3. The bone conduction audio device of claim 2, further comprising an I/O device that transmits the user's voice to an auxiliary device.

4. The bone conduction audio device of claim 1, further comprising a filter that attenuates one or more frequency ranges in the sound.

5. The bone conduction audio device of claim 1, further comprising a filter that attenuates the sound when the sound is above a particular decibel level.

6. The bone conduction audio device of claim 1, further comprising a filter that amplifies one or more frequency ranges in the sound.

7. The bone conduction audio device of claim 1, further comprising one or more hearing protection devices.

8. A bone conduction audio device for use with one or more hearing protection devices, the bone conduction audio device comprising:
one or more microphones that record sound;
one or more conduction transducers that transmit the sound to a user via a skeletal structure of the user; and
a support that secures the one or more conduction transducers in engagement with the skeletal structure of the user when the bone conduction audio device is worn by the user; and
one or more mounts for holding the one or more hearing protection devices;
wherein the user's ears are not obstructed by the one or more conduction transducers.

9. The bone conduction audio device of claim 8, further comprising an auxiliary microphone that records a user's voice.

10. The bone conduction audio device of claim 9, further comprising an I/O device that transmits the user's voice to an auxiliary device.

11. The bone conduction audio device of claim 8, further comprising a filter that attenuates one or more frequency ranges in the sound.

12. The bone conduction audio device of claim 8, further comprising a filter that attenuates the sound when the sound is above a particular decibel level.

13. The bone conduction audio device of claim 8, further comprising a filter that amplifies one or more frequency ranges in the sound.

14. The bone conduction audio device of claim 8, wherein the support comprises a resilient structure.

15. A method for providing auditory perception with a bone conduction audio device, the method comprising:
providing one or more conduction transducers that transmit sound to a user via a skeletal structure of the user, wherein a support secures the one or more conduction transducers in engagement with the skeletal structure of the user when the bone conduction audio device is worn by the user;
capturing sound via one or more microphones; and
transmitting the sound to the user with the one or more conduction transducers via the skeletal structure of the user;
wherein the user's ears are not obstructed by the one or more conduction transducers.

16. The method of claim 15, further comprising providing an auxiliary microphone that records a user's voice.

17. The method of claim 16, further comprising transmitting the user's voice to an auxiliary device with an I/O device.

18. The method of claim 16, further comprising attenuating the sound with a filter when the sound is above a particular decibel level.

19. The method of claim 16, further comprising amplifying one or more frequency ranges in the sound with a filter.

20. The method of claim 16, further comprising obstructing the sound at the user's ears with one or more hearing protection devices.
